(19) United States
(12) Patent Application Publication
(10) Pub. No.: US 2025/0288723 A1
(43) Pub. Date: Sep. 18, 2025

(54) METHOD FOR DECELLULARIZATION OF A TISSUE, DECELLULARIZED TISSUE MATRIX AND A SCAFFOLD FOR USE THEREOF IN TISSUE REPAIR

(71) Applicant: INSTITUTO DISTRITAL DE CIENCIA BIOTECNOLOGIA E INNOVACIÓN EN SALUD - IDCBIS, Bogotá (CO)

(72) Inventors: Luz Stella CORREA ARAUJO, Bogotá (CO); Ingrid Zulay SILVA COTE, Bogotá (CO)

(21) Appl. No.: 18/862,355

(22) PCT Filed: May 5, 2023
(86) PCT No.: PCT/IB2023/054676

§ 371 Date: Nov. 1, 2024

(30) Foreign Application Priority Data
May 6, 2022 (CO) NC2022/0005963

(51) Int. Cl. A61L 27/36; C12N 5/071

(52) U.S. Cl. CPC A61L 27/3687; C12N 5/0629; C12Y 301/21001; C12N 2501/73

(57) ABSTRACT

A method of decellularizing a tissue, the decellularized tissue matrix obtained by the method, and a scaffold comprising the matrix. The method of tissue decellularization comprises the steps of: providing a tissue sample, subjecting the sample to at least three cycles of freezing at −80° C. and thawing at 40° C., adding a hypertonic solution by constant stirring at room temperature for 4 hours and removing the solution, adding Trypsin-EDTA for 1 hour at 37° C. with stirring, storing the sample in a non-ionic surfactant for 10-12 hours, and subsequently removing the surfactant and incubating the sample with an endonuclease for 6 hours at 37° C.

### FIELD OF THE INVENTION

The present invention relates to a method of decellularizing a tissue, to the decellularized tissue matrix obtained by the method, and a scaffold comprising said matrix.

The invention has application in tissue engineering and regenerative therapy.

### PRIOR ART

Decellularization is a process used in biomedical engineering to isolate the extracellular matrix (ECM) of a tissue by removing the cells themselves and leaving an extracellular matrix scaffold of the original tissue, which can be used as a support for cell growth and proliferation in tissue regeneration.

Decellularized human skin has been used for a variety of medical procedures, including primarily wound healing, soft tissue reconstruction, and sports medicine applications.

The methods of tissue decellularization are characterized by long time requirements and methodologies that can degrade the tissue or matrix. For tissue to be used as a safe and effective tissue scaffold, it is necessary to ensure that it is free of bacteria and viruses, is completely decellularized and free of DNA to avoid graft rejection, and that it retains the functional properties of the native tissue from which it is derived, including biomechanical properties and the ability to function as an extracellular matrix.

Methods for producing decellularized tissue scaffolds or matrices already exist in the prior art.

WO2021176226A1 describes a method for producing a decellularized tissue scaffold comprising the steps of providing a pre-treated tissue sample, incubating the tissue with sodium dodecyl sulfate (SDS) or sodium deoxycholate, incubating in a hypotonic solution of Tris-EDTA (0.1%-0.1%) at least once between 30 minutes and 100 hours, and a hypertonic solution of Trypsin-NaCl (0.6%-9%) at least once between 1 and 48 hours, and/or subjecting the sample to at least one freeze/thaw cycle, and finally incubating the sample with a DNase I solution for 2-24 hours, thus obtaining a decellularized tissue scaffold. The method employs reduced levels of anionic detergent and avoids the use of animal-derived protease inhibitors to produce a tissue scaffold with favorable properties. However, long periods of time are used, which can damage the resulting matrix.

US2013028981A1 describes a method for producing a decellurized bio-prosthetic tissue. The method comprises the steps of providing a pre-treated tissue sample, contacting the sample with a first surfactant, contacting the sample with a nuclease enzyme solution and with a cleaning solution comprising a second surfactant (1% tri-n-butyl (TnBP)), a chaotropic agent or a mixture thereof to produce decellularized tissue, and contacting the decellularized tissue with a bio-burden reducing agent (1% peracetic acid) to produce the final bio-prosthetic tissue. However, the use of a second surfactant, in addition to the chaotropic agent, and the use of peracetic acid affects the final properties of the matrix.

US2015050247A1 describes decellularized extracellularized tissue-derived matrices (ECMs) and methods for generating and using the same. The method for generating a decellularized matrix includes the steps of: (a) subjecting the tissue to washes and hypertonic buffer; (b) subjecting the tissue to enzymatic proteolytic digestion with an enzyme such as trypsin; and (c) removing all cellular components from the tissue using a detergent solution including Triton-X-100 and ammonium hydroxide. However, they do not employ any endonuclease and, therefore, do not generate DNA strand breaks by hydrolysis. This directly affects the ability to obtain a tissue with a reduced or low level of residual DNA, which is necessary to obtain a tissue that does not produce rejection when used as an implant.

CN109675112A relates to a method for producing a decellularized dermal matrix of human origin. The method of preparation specifically comprises the following steps: taking stored skin of human origin, removing subcutaneous adherent fatty ingredients, cleaning and disinfecting with iodine and ethyl alcohol, then repeatedly soaking and washing the skin with a hypotonic solution, treating using a solution of Dispase Il overnight, removing the epidermis, and, after that, washing with normal saline, thus obtaining the dermis. This dermis is subsequently treated with hypertonic saline, washed with normal saline, and treated using a decellularization solution comprising trypsin, EDTA, Triton X-100, Na⁺, Cl⁻ and Ca⁺² in a dialysis device for 2 h-6 h, to obtain the decellularized dermal matrix. In this process, Dispase Il is used all night long, which can cause significant damage to the extracellular matrix, since this enzyme is capable of degrading fibronectin and collagen I present in the matrix. In addition, it employs Trypsin which can also affect the matrix. On the other hand, the procedure does not remove residual DNA as a result of cell lysis.

RU2717088C1 relates to a method of producing an acellular dermal matrix. The method comprises the steps of providing a pre-treated tissue sample, freezing the sample at a temperature of −80° C. and subsequently thawing, contacting the sample in a Trypsin-EDTA solution, and stirring at 100 rpm at 37° C. for 18 hours; then the sample is placed on a turntable at 170 rpm and subjected to sequential cyclic action of detergent solutions: 1% Triton X-100 solution for 2 hours and 4% sodium deoxycholate in combination with 0.002 M Na2-EDTA for 2 hours (repeated at least 5 times), incubation the sample with DNase I solution with stirring (100 rpm) at 37° C. for 4 hours, and finally the sample is exposed to 10% chlorhexidine bigluconate in phosphate buffer for 24 hours with solution change every 6 hours. The method allows to reduce the exposure time of decellularizing solutions, reducing the level of residual DNA in the tissue down to 60 ng/mg of tissue in a wet sample. However, the level of residual DNA is still considered high, so there remains a high risk of rejection when used as an implant. Additionally, exposure of the sample to Trypsin-EDTA for 18 hours can cause severe damage to the acellular matrix composition.

Therefore, there is a need for new methods of tissue decellularization to obtain completely acellularized matrices free of genetic material to reduce the complications of the use of this type of decellularized tissues in transplantation (inflammation, degradation, scarring, contracture, calcification, occlusion and/or rejection).

### DETAILED DESCRIPTION OF THE FIGURES

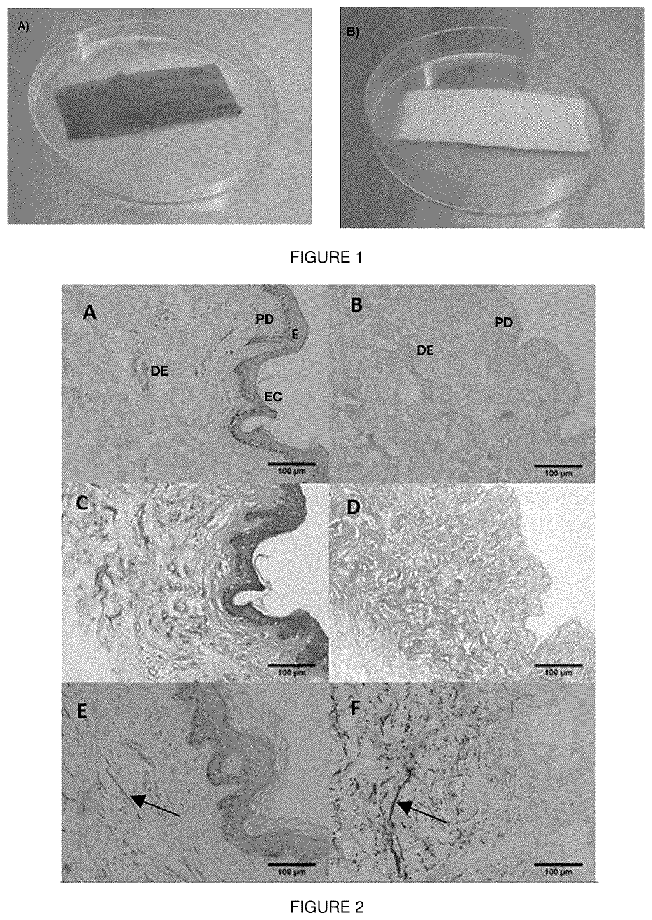

FIG. 1. Decellularization of human cadaveric skin. Skin from cadaveric donor (A), acellular dermis after the decellularization method (B) of the present invention.

FIG. 2. Evaluation of the skin decellularization process.

Hematoxylin and Eosin Staining of untreated skin (A) shows the epidermis (E), dermal papillae (DP), dermis (DE), and stratum corneum (SC), while the acellular dermis (B) shows only extracellular matrix and dermal papillae.

Masson's trichrome staining of the untreated skin (C) shows the regions identified in the hematoxylin and eosin staining, as well as the collagen fibers, and in the acellular dermis (D) the collagen fibers and dermal papillae are preserved.

Verhoeff-Van Gieson staining of untreated skin (E) and acellular dermis (F) shows elastin fibers (arrows), which are necessary for the elasticity of the new tissue. Images taken under light microscopy; scale bar: 100 μm.

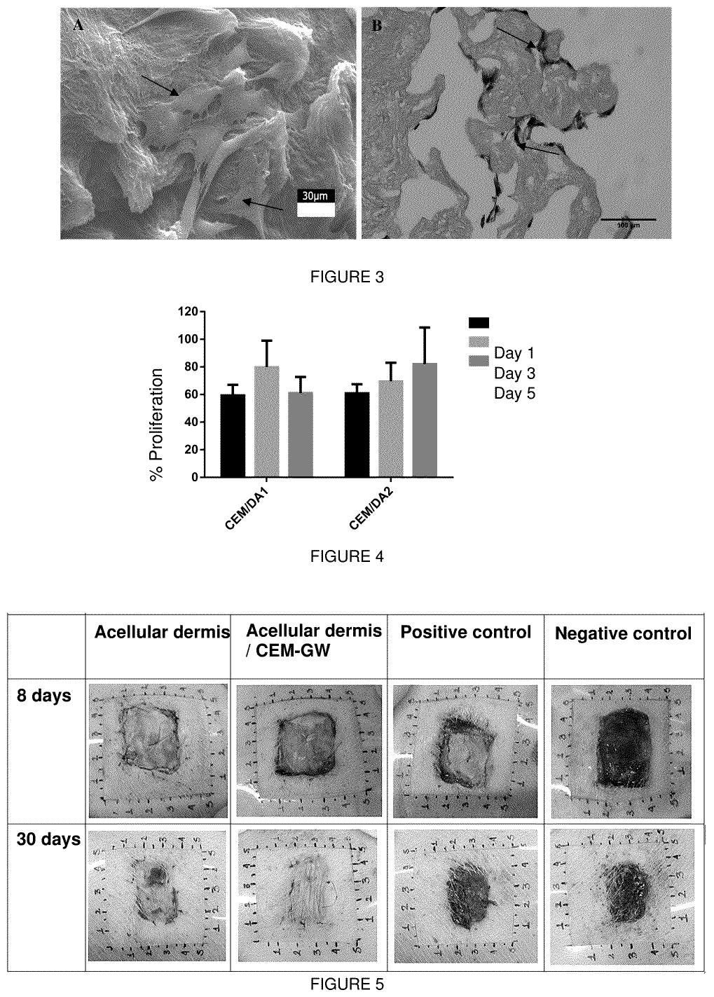

FIG. 3. Cell adhesion and proliferation capacity of mesenchymal stromal cells in the acellular dermis. Wharton's jelly mesenchymal stromal cells from the umbilical cord (CEM-GW) attached to the matrix surface were identified by scanning electron microscopy (A) and hematoxylin and eosin staining (B). Cells are indicated by the arrows. Scale bar=30 μm and 100 μm, respectively.

FIG. 4. Cell proliferation assay through resazurin. The percentage of CEM-GW cell proliferation on the scaffold (DA1 and DA2) was correlated with cell growth up to five days of culture, the values were normalized using the control group (cells only).

FIG. 5. Repair of skin lesions using the acellular dermis obtained by the method of the present invention, a positive control (commercial dermis), and a negative control (untreated lesion).

### DETAILED DESCRIPTION OF THE INVENTION

The present invention relates to a decellularization method wherein complete tissue acellularity is obtained in only one week of treatment. In turn, the structure of extracellular matrix proteins, including collagen and elastin, is preserved, as well as the preservation of dermal papillae, all of which are crucial components in skin repair therapy due to their ability to promote cell adhesion and growth.

These properties favor the integration of acellular tissue in the injured area, promoting the repair of full-thickness skin wounds (removal of epidermis and dermis) through the formation of a basement membrane capable of restoring the epithelial layer and the production of extracellular matrix proteins that form the new tissue.

In a first aspect, the present invention relates to a method of decellularizing a tissue, characterized in that it comprises the following steps:
a) providing a tissue sample;
b) subjecting the sample to at least three cycles of freezing at −80° C. and thawing at 40° C.;
c) adding a hypertonic solution by constant stirring at room temperature for 4 hours and removing said solution;
d) adding Trypsin-EDTA for 1 hour at 37° C. with stirring;
e) storing the sample in a nonionic surfactant for 10-12 hours and then removing said surfactant; and
f) incubating the sample with an endonuclease for 6 hours at 37° C.

In the method of the present invention, the exposure time to detergents that can deteriorate the extracellular matrix is reduced. In addition, stirring times that promote cell removal are used.

In addition, the exposure time to the endonuclease is longer in order to efficiently remove the DNA, which can generate immunorecognition in the patient.

First, the freeze/thaw stage allows the rupture of the membrane surrounding the tissue's own cells and in turn promotes the formation of intracellular ice crystals, favoring cell lysis or rupture. In addition, the repetition of this freeze/thaw cycle ensures cell membrane rupture and intracellular lysis.

Next, exposure of the tissue to a hypertonic solution, preferably sodium chloride at different concentrations (0.5 M and 1 M), promotes lysis of the cells (which are still preserved in the tissue) by osmotic shock, this process is complemented by periods of stirring in order to remove the cells located in the basement membrane that makes up the skin epithelium. Hypertonic solutions can maintain the structure of the basal layer, in this case the dermal papillae, and the functionality of the native matrix.

With subsequent exposure of the tissue to an enzymatic compound, in this case Trypsin-EDTA, cellular components are removed and cell-matrix junctions are disintegrated by cleavage of peptide bonds, this process is accompanied by agitation and temperature at 37° C. to promote the enzymatic activity of Trypsin.

It is important to highlight that one hour of exposure is sufficient to guarantee cell removal, considering that Trypsin is capable of eliminating proteins from the extracellular matrix, causing damage to its structure. This enzymatic process is crucial due to the complete separation between the epithelium and the dermis, which is noticeably evident once the treatment is completed.

By exposing the matrix to non-ionic surfactants, preferably Triton X-100, DNA-protein, lipid-lipid, and lipid-protein interactions are disrupted, which facilitates the complete removal of residues present in the matrix while maintaining the structure of native proteins such as Collagen and Elastin.

For the complete removal of the residual genetic material, product of cell lysis, an endonuclease, preferably DNAse type I, is subsequently used at 37° C., capable of generating the rupture of the DNA strands by hydrolysis. This reaction is used after treatment with the nonionic surfactant due to the porosity that this generates in the matrix, facilitating the infiltration of the endonuclease and, therefore, its hydrolytic activity, in addition to favoring the washing of the nonionic surfactants previously used. At this stage, exposure was standardized for 6 hours with inversion of the dermis every 3 hours to achieve greater effectiveness of the enzyme.

The use of DNase with a non-ionic surfactant has been shown to be essential for the complete removal of genetic material, achieving a DNA reduction of more than 95% after treatment. This parameter is crucial when assessing the quality of the acellular dermis, wherein DNA concentrations of each processed sample have been evaluated and values less than or equal to 4 ng/mg have been obtained, this concentration is considered an indicator of adequate removal of DNA residues in a biological scaffold, including the acellular dermis.

In another aspect, the present invention relates to a decellularized tissue matrix obtainable by the method of the present invention as described above. The decellularized tissue matrix has a DNA concentration of less than or equal to 4 ng/mg, which is advantageous, as discussed above.

In another aspect, the present invention relates to a scaffold comprising a decellularized tissue matrix as described above.

In a last aspect, the decellularized tissue matrix or scaffold of the present invention can be used in tissue engineering and regenerative therapy.

### Examples

### Example 1. Method of tissue decellularization

Human skin sections (10×5 cm²) supplied by the IDCBIS District Tissue Bank, stored in 85% glycerol, were washed in Petri dishes containing 15 mL of sterile 1× PBS. This stage was repeated once again.

Subsequently, they were placed in 500 mL sterile bottles and each sample was subjected to three cycles of freezing (−80° C.) and thawing (40° C.), wherein each cycle lasted 15 minutes. Then 200 mL of sterile distilled water was added to each vial and stored at 4° C. for two days.

Next, sterile distilled water was removed and 200 mL of a 0.5 M NaCl solution was added for 4 hours under constant stirring (150 rpm) at room temperature, then this solution was removed and 200 ml of 1 M NaCl was added for 4 hours under constant agitation, the solution was mixed into each sample and stored in sterile distilled water overnight at 4° C. The procedure of exposure to hypertonic solution at both concentrations (0.5 M and 1 M) was repeated once more.

The samples were then treated with 50 mL of 0.25% Trypsin-EDTA for one hour at 37° C. with constant stirring. They were then washed in 100 ml of sterile distilled water and transferred to a new sterile flask, wherein 150 mL of sterile distilled water was added to each flask and the sample was left under stirring for one hour, in order to remove Trypsin-EDTA residues.

Next, the samples were stored in 200 ml of 1% Triton X-100 for 10 to 12 hours at 4° C. Subsequently, the detergent was removed, and each sample was washed three times with 100 ml of sterile distilled water for five minutes, and then stored in Petri dishes containing 13 mL of DNase I. The sample was incubated at 37° C. for 6 hours, and inversion of each tissue was performed after the first three hours.

Finally, the samples were transferred to the previously used sterile vials containing 200 mL of sterile distilled water, which was replaced after 24 hours, and each sample was stored at 4° C. The final result is an acellular dermis.

The method of the invention reduces the exposure time to detergents that can deteriorate the extracellular matrix; in addition, stirring times that promote cell removal are employed. On the other hand, in the present method, the exposure time of the sample with DNase lis prolonged in order to efficiently eliminate the DNA, product of cell degradation and that can generate a risk of immunological rejection.

FIG. 1 shows variation in tissue shade after performing the decellularization method of the present invention. Visually, there are no major changes in its structure.

FIG. 2 shows how the decellularization process preserves fundamental structures for skin repair, including dermal papillae and extracellular matrix proteins such as collagen and elastin.

FIG. 3 shows the in vitro results of the evaluation of the ability of the acellular dermis to promote cell adhesion and growth. Among the many advantages of the acellular dermis are its important extracellular matrix content and biomechanical properties that considerably favor cell growth, which are crucial in the process of repairing a lesion.

FIG. 4 shows the results of the cell proliferation assay through resazurin. The percentage of CEM-GW cell proliferation on the scaffold (DA1 and DA2) was correlated with cell growth up to five days of culture, the values were normalized using the control group (cells only). These results indicate that the dermis favors cell proliferation.

### Example 2. Repair of Skin Lesions Using the Acellular Dermis Obtained by the Method of the Present Invention

Acellular dermis was used for skin lesion repair in a porcine bio-model. The area of each wound was delimited and the dermal substitutes were implanted in the bio-model.

The results after thirty days of treatment indicated complete integration of the acellular dermis into the injured area, absence of inflammation, granulation tissue formation, re-epithelialization, minimal shrinkage and absence of scarring.

Additionally, the acellular dermis was recellularized with Wharton's jelly mesenchymal stromal cells (CEM-GW) and implanted into the bio-model lesion, wherein complete lesion closure with minimal shrinkage was evident.

FIG. 5 shows the results obtained. These results were compared with the positive control corresponding to an acellular dermis of commercial use, obtaining better results in the lesions treated with the dermis of the present invention, unlike the negative control, i.e., untreated wound, in which a low repair was observed with evident contraction and formation of fibrin scab.

These evidences demonstrate the potential of the matrix of the present invention as a bio-compatible skin substitute with skin repair capability.

1. A method of decellularizing a tissue, comprising the following steps:
a) providing a tissue sample;
b) subjecting the sample to at least three cycles of freezing at −80° C. and thawing at 40° C.;
c) adding a hypertonic solution by constant stirring at room temperature for 4 hours and removing said solution;
d) adding Trypsin-EDTA for 1 hour at 37° C. with stirring;
e) storing the sample in a nonionic surfactant for 10-12 hours and then removing said surfactant; and
f) incubating the sample with an endonuclease for 6 hours at 37° C.

2. The method according to claim 1, wherein the hypertonic solution is a NaCl solution.

3. The method according to claim 1, wherein the concentration of hypertonic solution is between 0.5 M and 1 M.

4. The method according to claim 1, wherein step c) is repeated at least once.

5. The method according to claim 1, wherein the non-ionic surfactant is 1% Triton X-100.

6. The method according to claim 1, wherein the endonuclease is DNase Type I.

7. The method according to claim 1, wherein the method further comprises one or more steps of rinsing the tissue.

8. A decellularized tissue matrix obtained by the method according to claim 1.

9. The decellularized tissue matrix according to claim 8, wherein the DNA concentration is less than or equal to 4 ng/mg.

10. A scaffold comprising a decellularized tissue matrix according to claim 8.

11. A method for tissue engineering and regenerative therapy comprising using the decellularized tissue matrix according to claim 8.
